(19) United States
(12) Patent Application Publication
(10) Pub. No.: US 2020/0160956 A1
(43) Pub. Date: May 21, 2020

(54) Methods and Systems to Support Health Outcome Improvement with Strategic Reduction in Economic Spend for Health Benefits

(71) Applicant: Troy Roth, Prosper, TX (US)

(72) Inventor: Troy Roth, Prosper, TX (US)

(21) Appl. No.: 16/686,867

(22) Filed: Nov. 18, 2019

Related U.S. Application Data
(60) Provisional application No. 62/768,282, filed on Nov. 16, 2018.

(51) Int. Cl. G16H 20/00; G16H 10/60; G06Q 30/06; G06Q 30/08; G16H 40/20; G16H 15/00; G06F 3/0482

(52) U.S. Cl. CPC G16H 20/00; G16H 10/60; G06Q 30/0641; G06F 3/0482; G06Q 30/0633; G16H 40/20; G16H 15/00; G06Q 30/08

(57) ABSTRACT

The disclosed principles provide a platform that facilitates for healthcare consumers the determining their healthcare needs and goals, identifying and connecting Care Team members to meet those needs/goals, determining the costs of treating the needs/goals, providing a schedule for meeting those needs/goals, monitoring the progress of achieving those needs/goals, determining whether the needs/goals are being met, identifying any additional or related needs/goals required to meet the original needs/goals, identifying barriers to achieving any of the needs/goals, and providing resources for overcoming those barriers. The platform consumes data to identify the populations' medical and preventative needs. A Longitudinal Care Plan and best service locations are created to set a baseline schedule and provide measurable care cost based on known requirements. The platform will capacity-plan based on in-network providers and service locations matched to purchasers' and patients' requirements, and if any tasks are unmet, the platform will solicit bids from other providers to match required services.

### RELATED APPLICATION

This disclosure claims the benefit of U.S. provisional patent application Ser. No. 62/768,282, filed Nov. 16, 2018, which is incorporated herein in its entirety for all purposes.

### TECHNICAL FIELD

This disclosure relates to the field of health benefits and coverage, and more particularly to purpose-built health networks designed to achieve improved clinical and financial outcomes to any given health purchaser goals and preferences. This enablement affords any willing health purchaser to purchase directly from healthcare service providers for memberships health needs while refining the science of patient activation and introducing a barrier-free healthcare benefit package customized to memberships health burdens and other needs.

### BACKGROUND

Due to government funding via the Health Information Technology for Economic and Clinical Health (HITECH) Act in 2009, we have witnessed many advancements in health information technology solutions merging health information with quality, outcomes and patient safety within the U.S. healthcare market; however, we have not realized pervasive change in our economic spend trajectory that is fundamentally required for the U.S. to remain competitive in the world economy. Most midsized to large multi-tenant integrated delivery networks manage enormous clinical technology stacks; yet they lack the ability to assess if their owned resources can deliver all services required for a given population while maintaining the quality and outcome standards for which their brand is recognized. As detailed below, the disclosed principles provide for full capacity management with scheduling services and ‘task-based’ activity-based costing fundamentals.

The Patient Protection and Affordable Care Act (PPACA) was passed in 2010. PPACA requires health insurance companies in the U.S. to increase insurance coverage for pre-existing conditions, and mandated medical loss ratios between 80-85 percent of premium dollars on healthcare quality improvement beginning in 2011. The final rule also set forth direction to states to apply Essential Health Benefits (EHB), Minimal Essential Coverage (MEC), and certification of Qualified Health Plans (QHP), including Small Business Health Options Program (SHOP) exchanges. The gains in quality healthcare services or improved premium financial management expected per PPACA design have not been realized and this fact is most evident in the lack of options and elections with the SHOP exchanges. Many small employers are eligible for the Small Business Health Care Tax Credit per the National Association of Insurance Commissioners (NAIC) recommendations, and the regulation called out 5 quality improvement activities demonstrated within published evidence-based practices that health plans may contribute toward improved care and outcomes may be included in the 80-85 percent standard.

An Executive Order (13813) issued by President Trump on Oct. 12, 2017 led the Department of Labor (DOL) to make changes in Final Rule 29 C.F.R. Part 2510 RIN 1210-AB85 to make changes to the definition of “Employer” under Section 3(5) of ERISA—Association Health Plans published in the Federal Register on Jun. 21, 2018. This change will help sole proprietors and small businesses afford health benefits and coverage for their employees via the formation of an Association Health Plan (AHP). Changes also positively impact AHP's with reduced regulatory restrictions, administrative costs, and strengthening of bargaining power to qualify for more insurance options and potentially afford them the ability to self-insure their insurance risk. Each of the aforementioned benefits will help curtail unnecessary cost to obtain health insurance.

Fundamentally, we are currently leveraging Direct Primary Care physician which are accustomed to providing 24/7/365 access to care by each patient's primary care physician who knows their patients better than indirect care teams. These physicians are not as rushed to move to the next patient, moreover, completely focused on delivering the best care and meeting 100% of the required care plan tasks to improve and/or stabilize current chronic illnesses.

### SUMMARY

Candidly, the U.S. healthcare delivery system requires perverse change in order to enable the U.S. economy to compete globally. Healthcare expenditures are the leading uncontrollable expense reported by Fortune 500 CFOs. However, it is not in the best economic interest of the major players in the U.S. system to disturb the current model because it introduces risk into their strategic business models. A primary goal of the disclosed principles is to move more money from third-party non-value-added service providers directly to care providers to directly enhance patient care. The systems/methods to implement the disclosed principles employ a combination of Association Health Plans, Health Reimbursement Arrangements, Healthcare Savings Accounts and Multiple Employer Programs outlined in ERISA. Key components and/or principles of a system and/or method in accordance with the platform(s) include:
Dynamic and automated healthcare needs assessment engine that pairs known and required healthcare services with highly available care givers and service providers who have a history of producing quality outcomes via healthcare purchaser-centric virtual brokerage tool.
Affords multiple healthcare service purchasers to band together and spread risk while reducing premiums via legislation change on their progression to self-insuring coverage or mutual ownership.
Healthcare needs-driven services auction connecting purchasers to sellers enabling administrative simplification and care warranties within purpose built, quality network formations.
Supply chain-principled aggregation of all healthcare needs into evidence-based treatment tasks which are attributed to appropriate healthcare service sellers based on scope of practice.
Reimbursement service capability designed to individual achievement of all of evidence-based tasks dictated by endorsed treatment plans, while rewarding/penalizing service providers for these quality-, outcome- and cost-based achievements.
Artificial Intelligence/Machine Learning to support health purchasers and membership/patients' preferences/tendencies to anticipate care needs, changing health conditions (stage and status), new business and most importantly—patient activation improvements.

The disclosed principles support healthcare purchasers' desire to pay for higher quality care at a more reasonable cost by enabling healthcare sellers to coordinate and manage care via the most efficient and effective providers, in the most appropriate care setting and at the most modest cost. A backend technology system in accordance with the disclosed principles formulates the best benefit and coverage via either direct arrangements or existing state endorsed health plans that match known required services of the healthcare purchaser's population. Commercially viable, third-party risk stratification is executed to provide past medical claim history to extrapolate illness burden, disease staging/state and projected spend under any former health plan contract and other clinical and financial constructs. This information will be coalesced with healthcare purchasers' objective goals and required quality, outcome, and spend expectations for matching to a provider registry of ambulatory licensed practitioners, and ancillary, acute and subacute providers within the desired network. The next step will merge in membership care consumption preferences and appropriate surveys to better understand threats to patient activation and other barriers to care which threaten their ability to fulfill requirements of both medical professional and self-care treatment plans.

The disclosed principles provide for purpose-built, customizable longitudinal care plans influenced by social determinates, all other barriers to service, care consumption preferences and health literacy/numeracy of membership. The platform considers membership deficiencies including access and social determinants of health to be positively influenced by the accountable dynamic network selected by healthcare purchasers.

A tactical assembly of the purchasers' population(s) as proposed are matched to qualified care providers at an individual task-based level enabling providers to individually state a defined scope of practice. Qualified providers can exist within existing Accountable Care Organization (ACO)/Integrated Delivery Networks (IDN) collaboratives or come from new strategic partnerships via a marketplace bidding process where healthcare providers from outside a given collaborative can Opt-In based on acceptance of purchasers' requirements of participation and meeting purchasers' outcomes and cost standards (e.g. quality, care setting, patient/non-clinical care giver satisfaction, location, etc.). All bids are based on available capacity and member care acquisition preferences.

The disclosed principles provide for care collaboration technical capabilities to host group level as well as 1:1 interaction via internet capable devices intended to support any member of care team, patient, member or any other known authorized patient advocates. It also supports patient-centric administrative processes, including access to providers, appointment setting, out-of-pocket estimates by selected cite of service, self-care supplies, equipment, Durable Medical Equipment (DME), and Pharmacy Benefit Manager (PBM) links for mail order and price shopping. Additional capabilities include: navigation/wayfinding and online bill pay, and auto deduction from a Health Savings Account (HSA), Flexible Spending Account (FSA), Medical Savings Account (MSA) and/or Health Reimbursement Account (HRA) based on member authorization.

The disclosed principles propagate a single datastore that aggregates quality and outcome information in a concise method that could be used to support Long Term Tort Reform, proving or disproving healthcare providers followed appropriate evidence-based longitudinal care plans. Patient safety would be positively influenced as well.

The disclosed principles provide for a care team engagement and governance model designed to place patients' interest above profit or service level reimbursement. This process and/or associated platform must be a physician led design with a well-managed communication strategy. The output of this process or platform will be task-based measures assigned to individual care team actors and/or patients or their advocates to create the denominator of the Health Barometer. These engagement and outcome goals drive workflow and services via the disclosed care consumption program to increase the quality and outcomes to each individual's care that is necessary to properly manage their known illness burden(s). Quality standard, health outcome disease staging and progression measures are included, as well as an evaluation of patient quality of self-care and identification of new or unremedied health literacy/numeracy and/or social determinates.

### BRIEF DESCRIPTION OF THE DRAWINGS

The invention(s) may best be understood with reference to the following detailed description and related drawings which illuminate the disclosed principles, and in which:

FIG. 1 illustrates a block diagram illustrating how a prospective patient or user may interact with a platform and related process in accordance with the disclosed principles;

FIG. 2 illustrates a block diagram of an exemplary process for health plan prospects that are “Shoppers” to navigate on a platform in accordance with the disclosed principles;

FIG. 3 illustrates a block diagram of an exemplary process of health plan prospects exemplary that are “Direct Contract” to navigate on a platform in accordance with the disclosed principles;

FIG. 4 illustrates is an exemplary process for a health plan purchaser to select coverage options for submission to healthcare sellers for bidding using a platform as disclosed herein;

FIG. 5 illustrates an exemplary list of questions that may be asked of a prospect on the Health Purchasers Goals page(s) illustrated in FIG. 1;

FIG. 6 illustrates an exemplary list of questions and options that may be provided on the Health Purchaser Profile page(s) illustrated in FIG. 1;

FIGS. 7A & 7B illustrate an exemplary list of the questions indexed as patient preferences on a platform in accordance with the disclosed principles;

FIG. 8 illustrates an exemplary process for compiling and submitting health plan proposals for bidding by healthcare sellers, using a platform as disclosed herein;

FIG. 9 illustrates is a diagram having a process flow for determining the primary care team and related provider costing for an exemplary purchaser's healthcare plan via a platform as disclosed herein;

FIG. 10 illustrates a diagram having a process flow for developing an exemplary incentive/disincentive model for using in health plan cost determining as performed on a platform implemented in accordance with the disclosed principles;

FIG. 11 illustrates detail breakouts from three steps of the process illustrated in FIG. 10;

FIG. 12 illustrates a diagram showing various access portals available to healthcare sellers via a platform as disclosed herein;

FIG. 13 illustrates an exemplary list of the de-identified membership/patient dataset available to Care Team applicants for Care Team applicants' bidding for health plans via a platform as disclosed herein;

FIG. 14 sets forth the expectations and desires of key healthcare actors in a system or method implemented in accordance with the disclosed principles;

FIG. 15 illustrates the needs-based and capacity-based dynamic capabilities of a system or method (platform/network) implemented in accordance with the disclosed principles;

FIG. 16 illustrates exemplary actions of task level, activity-based care teams (actors) in a system or method implemented in accordance with the disclosed principles;

FIG. 17 illustrates an exemplary Longitudinal Care Plan (LCP) for a patient/member created by a system or method (platform/network) implemented in accordance with the disclosed principles; and

FIG. 18 illustrates additional time/date-based information for a portion of the exemplary Longitudinal Care Plan illustrated in FIG. 17 for a patient/member created by a system or method (network) implemented in accordance with the disclosed principles.

### DETAILED DESCRIPTION

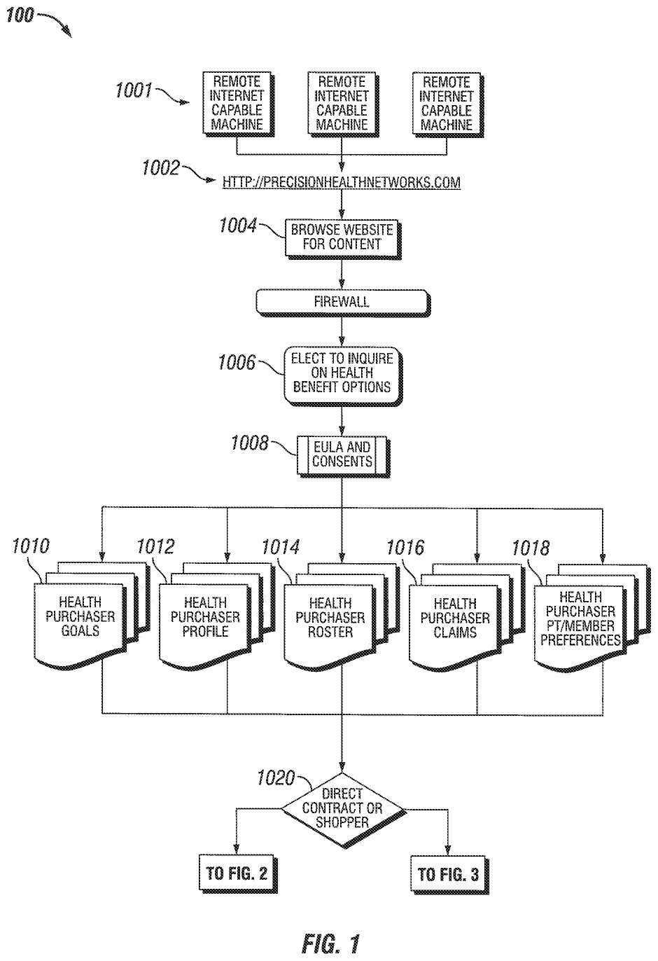

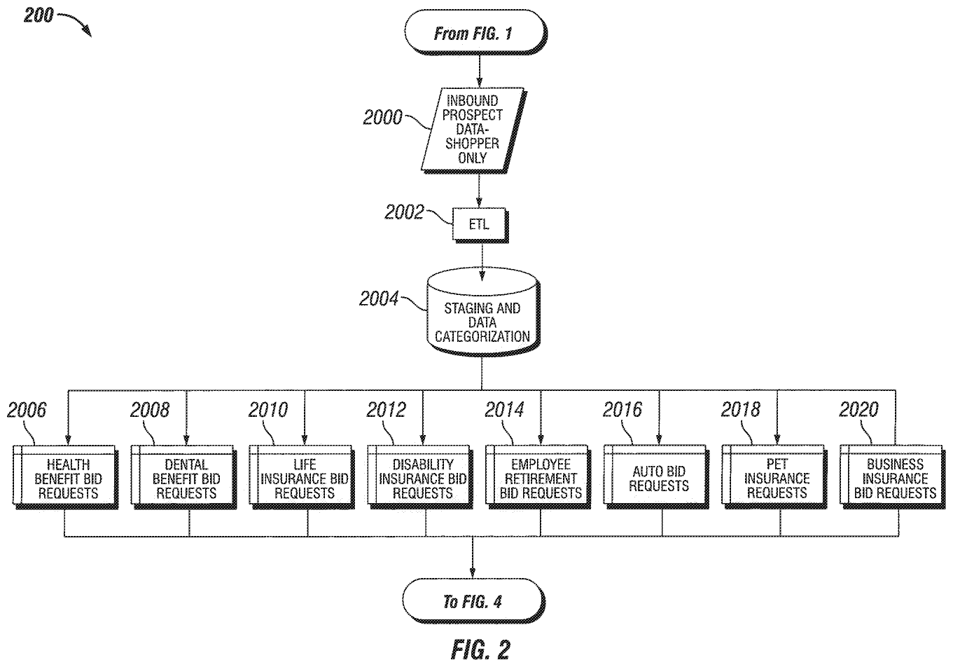

FIG. 1 illustrates a block diagram 100 illustrating how a prospective patient or user may interact with a platform and related process in accordance with the disclosed principles. The user will navigate to the platform from an internet capable machine 1001 via a hyperlink 1002 using a supported internet browser. Use of the platform is provided via a website 1004. If a prospective user elects to inquire about Health Benefit Options 1006, they will be required to accept terms of the End User Licensing Agreement (EULA) and provide consent 1008 so that “Precision Health Networks” (or other appropriate entity name), the administrator of the platform, may submit bid requests on the health purchaser's behalf to available domestic or state licensed health plans.

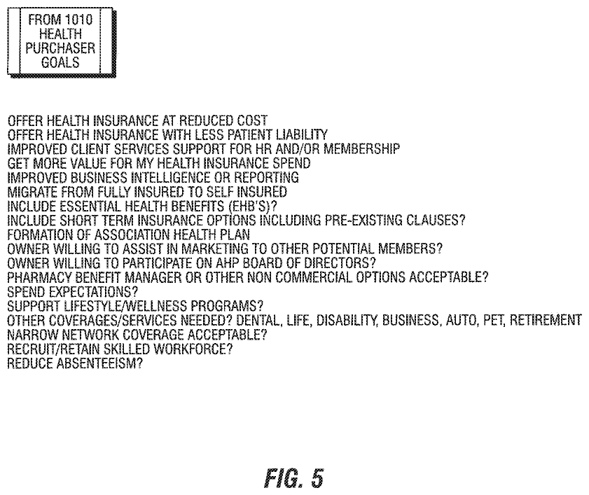

When the user selects Health Purchasers Goals 1010, they will be prompted to respond to a few very simple questions. Looking briefly at FIG. 5, illustrated are exemplary question that may be asked of the prospect on the Health Purchasers Goals 1010 page(s), such as: want to offer health insurance for themselves and/or their employees that are presently not covered through a group health plan, looking for better pricing options, looking for better benefits and coverage they receive from an existing health plan, looking for value-based (or custom) benefits and coverage to match their memberships existing illnesses, need better benefits and coverage to compete for skilled labor, keep existing/known doctors in network, gather a clear understanding of employer based spend tolerances, desires to form an Association Health Plan or other legal entity formations which can be used to join purchasing power to achieve better rates, other alternatives such as HRA and HSA programs, prefer fully insured (no financial risk) or self-insured (includes financial risk), adequacy of business intelligence or reporting for employee benefit costs. These questions are simply exemplary, and no limitation to any particular questions is intended. Based on the responses to questions, the prospective purchase will be routed to the next steps/pages in the process, as illustrated in FIG. 1.

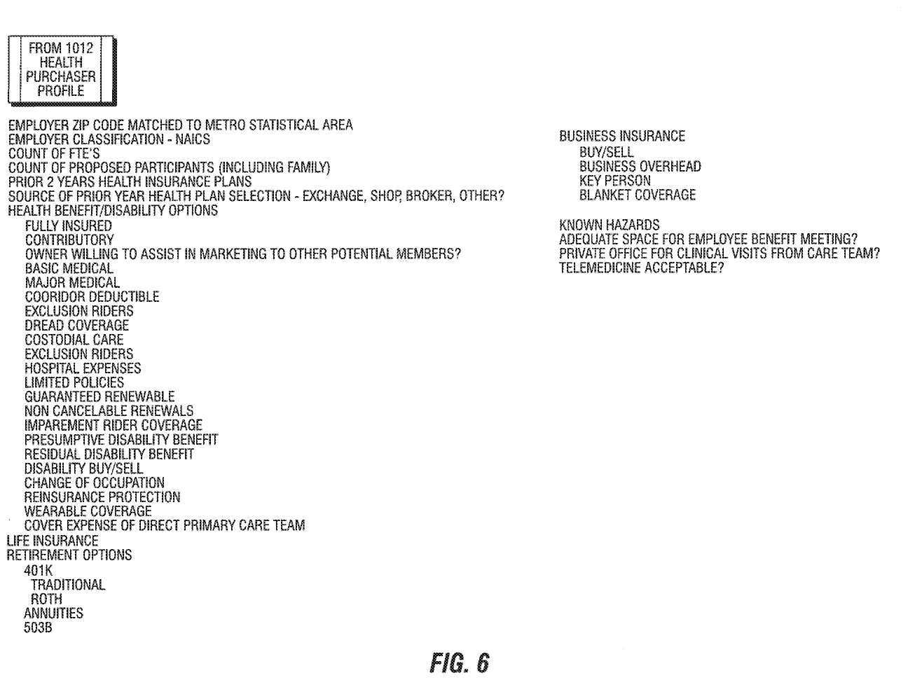

In the Health Purchaser Profile 1012, the prospect will be routed to a web form and/or they will be able to select and download the form for later completion. Exemplary contents of the web form for the Health Purchaser Profile 1012 are illustrated in FIG. 6. As illustrated in FIG. 6, this profile will have varying required fields based on their selections of coverages desired. Required fields may include the business or employer postal ZIP code which will be mapped by the platform to its corresponding Metropolitan Statistical Area. The platform will use the Metropolitan Statistical Area for benefit discovery coverages in that area. A lookup table will be propagated to allow the prospect to select the appropriate NAICS employer classification. This information is needed should the prospect elect to consider forming an Association Health Plan, HRA or migration to self-insured status. A count of FTE's is required for categorization as well as knowledge sharing for the health purchaser that group-based plans are contributory plans and require at least 75% participation. Count of proposed participants are required for categorization and insurance bids. If the health purchaser has had health coverages over the last 2 plan years, the platform can request health plan information and product selected to assist in matching network benefits/coverages and providers to prior coverage. Source of purchase for prior years health plans may be optional and aids the platform's decision support system in matching purchasers' goals to appropriate networks.

Health Benefits/Disability Options may be optional and used by the platform's decision support to best match purchasers' desires and goals to appropriate benefits and coverage. If a health purchaser is Fully Insured, the end user will be asked if they would consider ‘Self Insured’ options if pricing and outcomes are favorable to spend tolerance. Contributory plan is used by the platform to match likelihood of if the health purchaser met the requirements of participation and matching logic to appropriate benefits and coverages available. Business “Owner willing to assist in marketing” is optional and only pertains to those prospects who answered Yes to Health Purchaser Goals that they ‘are willing to form AHP’. The U.S. Department of Labor has very specific rules that must be followed including Board of Director formation and administration. Basic Medical, Major Medical, Corridor Deductible, Exclusion Riders, Dread Coverage, Custodial Care, Exclusion Riders, Hospital Expenses, and Limited Policies are all different types of specialized coverages that the platform will solicit bids from if prospect answers “Yes” to.

Insurance law has many specific types of protections and coverages that can supersede the main policy, including but not limited to Guaranteed Renewable, Non-Cancelable Renewals, Impairment Rider Coverage. Due to most health purchasers lack of understanding insurance law, the platform will also provide full definitions, helpful tips and recommendations and will bid these options out if the prospect so desires. If Disability coverage is selected from FIG. 2, Disability Insurance Bid Request, the prospect will be asked if they want bids for Presumptive Disability Benefit, Residual Disability Benefit, Change of Occupation, Any Occupation or Own Occupation, and the platform will also provide full definitions for each. Disability Buy/Sell requires “Yes” to Disability as well as “Yes” to Business Insurance Bid Request 2020, as illustrated in FIG. 2 and discussed in further detail below. Full definition will be available for the prospect. Reinsurance Protection allows for health purchaser to buy addition ‘stop-loss’ insurance to mitigate risk within the selected plan. Wearable coverage is a business expense if the health purchaser wants to purchase biometric devices to help employees manage lifestyle or wellness goals with the care team. Coverage expense for Direct Primary Care Team is a shift from commercial managed care networks to Direct Primary Care physicians who are directly compensated on a capitated model; they are responsible for 24/7/365 coverage needs of the health purchasers' membership.

Returning to FIG. 6, Life Insurance options via health purchaser or employer are often times less restrictive than individual policies and will include Group Life for the actual employee which pays out a multiple of your annualized salary and Dependent Group Life for dependents which pay out at fixed dollar amounts. The platform will execute based on health purchaser elections and include parameters selected by the end user. Retirement Options includes 401K options of Traditional 401K matching selection and Roth 401K tax sheltered options based on type selected. Traditional 401K are tax sheltered up to a maximum contribution with catch-up features for employees over the age of 50 upon contribution; Roth 401K are tax shelters upon withdrawal without fees based on your age at withdrawal. Annuities options are available as well, usually governed by Life Insurance rules and have Tax benefits. Options include Fixed with limited downside risk and are conservative and guarantee a minimum interest rate, Index-linked which are less conservative however, they historically have higher yields however, no guaranteed minimum interest rate is available and lastly Variable which are similar to mutual funds without minimum interest rate and you can lose some or all of your money invested. 503b plans could be available for some non-profit organizations.

Also in FIG. 6, Business Insurance, if elected as “Yes”, the purchaser will have options of Buy/Sell which is a type of coverage for business owners and their heirs if the owner dies unexpectedly. Business Overhead is also available, which covers short term periods typically 12-24 months designed to allow small business owners manage expenses while they are disabled and unable to work. Key Person is also available, which is a type of insurance that protects small businesses if the named insured person dies. Blanket coverage is also available, which is a type of insurance that protects commercial enterprises that might have one or numerous locations coverage against loss.

Also in FIG. 6, Known Hazards is also available, which is a type of risk that increases the possibilities of loss. Certain job roles are hazardous and make it difficult to qualify for individual and sometimes group insurance. Adequate space for Employee Benefit meetings are questions surrounding if the health purchaser actually acquires coverage from the platform, the company would like to know if adequate space exists to discuss benefits and coverage with the employees. Private Office for Clinical Visits from Care Team more surrounds if the health purchasers would like and use clinical provider visits at their place of employment; this is an atypical benefit that is used to increase patient or member engagement when they do not make time to go to their physician's office. Lastly, in this illustrated embodiment, Telemedicine Acceptable is a benefit option to help capture rates for those most unlikely to go to their physician office for visits.

Turning back to FIG. 1, on the Health Purchaser Roster 1014 page, prospective health purchasers are asked to provide a full roster of all employees and dependents expected to obtain coverage. Information will include, for example, Names, Home address, City, State, ZIP code, Sex, Date of Birth, Height, Weight, presence of any dread disease in last 5 years and if current pregnancy exists.

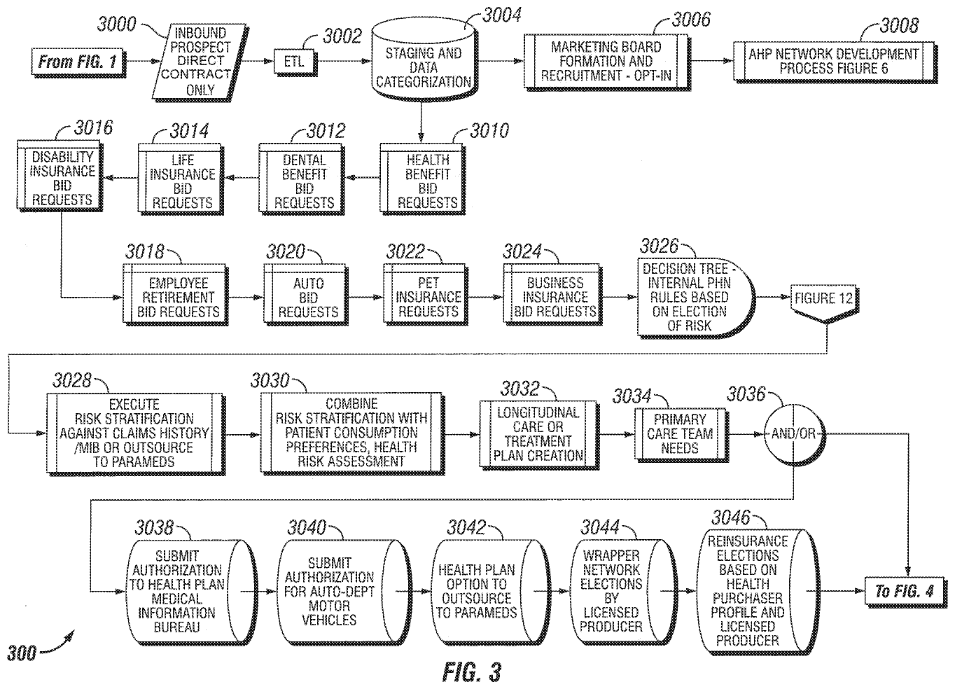

Also in FIG. 1, the prospect navigates to a Health Purchaser Claims 1016 page, for providing prior 2 years of claims history, which may be provided, if applicable, in standard ANSI 8371 and P format. These text files can be uploaded securely to the website for risk stratification, patient preferences 1018 (discussed in more detail below), and habits to calculate disease/illness burden and trajectory, which will be used by the platform to create longitudinal evidenced-based care plans and care team attribution and special needs. After preferences are entered, the prospect makes an election to the platform of whether they are a “Shopper” or “Direct Contract.” If the prospect is a “Shopper” navigation on the platform moves to FIG. 2, and if the prospect is a “Direct Contract” navigation on the platform moves to FIG. 3.

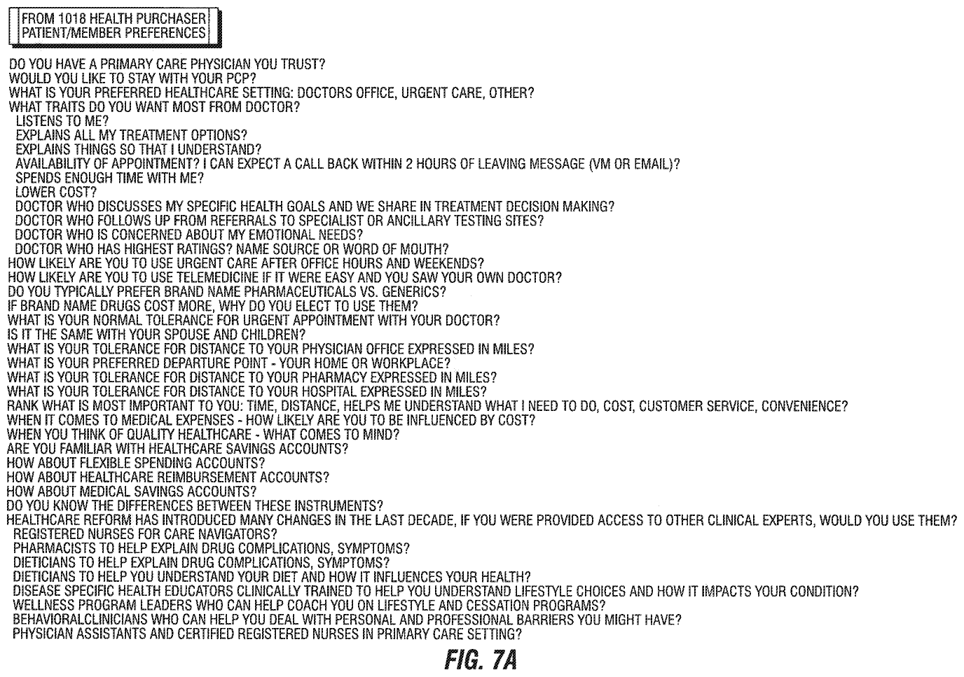

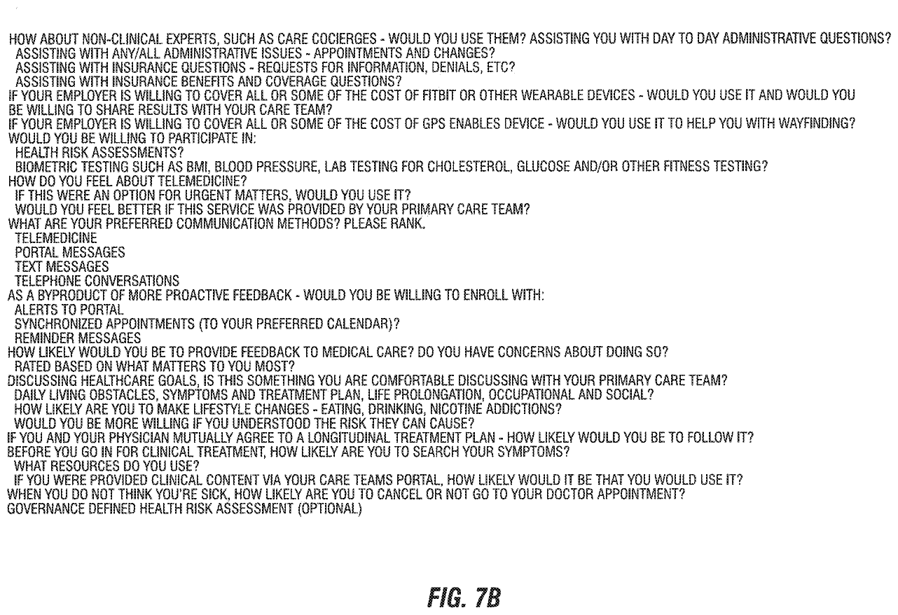

In FIG. 1, the Health Purchaser Patient/Member Preferences 1018 page has an extensive list of questions for patients including employees and dependents that the platform utilizes to score likelihood patients will seek out care from appropriate care givers who are incentivized to deliver 100% of each individuals' care needs and match patients/members to appropriate care teams based on preferences. Turning briefly to FIG. 7A and FIG. 7B, together they illustrate is an exemplary list of the questions indexed as patient preferences. These may include, but are not limited to, Do you have a Primary Care Physician (PCP_you trust? (Yes/No question) Would you like to stay with your PCP? (Yes/No Question with prompt for Physician Name and City from which they practice). What is your preferred healthcare setting: Doctors Office, Urgent Care or Other—If Other, please define and why? What Traits do you want most from your Doctor? This is a ranking question in which the patient/member can only select along a scale, such as 1-12. These may include: Listens to me? Explains all my treatment options? Availability of appointment? I can expect a call back within 2 hours of leaving message? Spends enough time with me? Lower Cost? Doctor who discusses my specific health goals and we share in decision making? Doctor who follows up from referrals to specialist or ancillary testing sites with results? Doctor who is concerned about my emotional needs? Doctor who has the highest ratings? (Define where ratings are captured) Named Doctors from Word of Mouth? How likely are you to use Urgent Care after office hours and weekends? How likely are you to use Telemedicine if it were easy and you saw your own Doctor? Do you prefer brand name pharmaceuticals versus generics? If brand name cost more, why do you elect to use them? What is your normal tolerance for urgent appointments with your Doctor? Is it the same with your spouse and children? What is your tolerance for distance to your physicians' office in miles? What is your preferred departure point—your home or workplace? What is your tolerance for distance to your pharmacy expressed in miles? What is your tolerance for distance to your hospital expressed in miles? Rank top to bottom what is most important to you: time, distance, helps me understand my treatment plan, cost, customer service or convenience? When it comes to medical expenses—how likely are you to be influenced by cost? When you think of quality healthcare—what comes to mind? Are you familiar with Healthcare Savings Accounts? How about Flexible Spending Accounts? How about Healthcare Reimbursement Accounts? How about Medical Savings Accounts? Do you know the differences between these instruments? Healthcare Reform has introduced many changes in the last decade, if you were provided access to other clinical experts, would you use them? Registered Nurses for Care Navigators? Pharmacists to help explain drug complications and symptoms? Dieticians to help you understand your diet and how it influences your health? Disease specific health educators clinically trained to help you understand lifestyle choices and how it impacts your condition(s)? Wellness Program leaders who can help you coach you on your lifestyle and cessation programs? Behavioral clinicians who can help you with anxiety, stress and other behavioral conditions you might have? Social Workers who can help you deal with personal and professional barriers you might have? Physician Assistants and Certified Registered Nurses in primary care settings? How about non-clinical experts, such as care concierge's—would you use them? Assisting you with day to day administrative questions? Assisting with any/all administrative issues—Appointments and changes? Assisting with insurance questions—requests for information, denials, etc.? Assisting with insurance benefits and coverage questions? If your employer is willing to cover all or some of the cost of FitBit or other wearables devices—would you use it and would you be willing to share results with your care team? If your employer is willing to cover all or some of the cost of GPS enabled devices—would you use it to help you with Wayfinding? Would you be willing to participate in: Health Risk Assessments? Biometric testing such as BMI, Blood Pressure, Lab testing for Cholesterol, Glucose and/or other fitness testing? How do you feel about Telemedicine? If this were an option for urgent matters would you use it? Would you feel better if this service was provided by your primary care team? What are your preferred communication methods: Telemedicine, Portal messages, Text messages, Telephone conversation? As a byproduct of more proactive feedback—would you be willing to enroll with: Alerts to Portal?, Synchronize appointments to your preferred calendar? Reminder messages? How likely would you be to provide feedback to medical care? Do you have concerns about doing so—if so why? Rated based on what matters to you most? Discussing Healthcare Goals, is this something you are comfortable discussing with your primary care team: Daily living obstacles, symptoms and treatment plan, life prolongation, occupational and social? How likely are you to make lifestyle changes—eating, drinking, nicotine addictions? Would you be more willing to change if you understood the risk they can cause? If you and your physician mutually agree to longitudinal treatment plan—how likely would you be to follow it? Before you go in for clinical treatment, how likely are you to search your symptoms? What resources do you use? If you were provided clinical content via your care teams portal, how likely would it be that you would use it? When you do not think you're sick, how likely are you to cancel or not go to your doctor's appointment?

Turning back to FIG. 2, illustrated is a block diagram of an exemplary process 200 for health plan prospects that are “Shoppers” to navigate on a platform in accordance with the disclosed principles. Since the prospect has elected that they are a “Shopper,” Inbound Prospect Data—Shopper Only step 2000 excludes prospects that identify themselves as interested parties in forming an Association Health Plan or AHP (these are discussed with reference to FIG. 3). Prospects will interact with the webpage via either fillable form-based manual keying of required and/or requested information, or downloading form(s) independently and uploading completed forms via firewall protected interface. The ETL or Extract, Transform and Load phase 2002 ensures web form mapping instructions and user error identification as the user interacts with the form. Users have the ability to download table formats and upload the completed forms. The ETL process will detect table identification and error handling instructions and propagate any errors back to the user. Staging and Data Categorization 2004 takes place after each webpage form is completed and/or uploaded from the prospect. Based on responses from the prospect, one to many of the following benefit requests will execute: Health Benefit Bid Request 2006 is expected to be the primary bid request and data attributes from FIG. 1 pages: Health Purchaser Profile 1012, Health Purchaser Roster 1014, Health Purchaser Claims 1016, and will be properly aggregated with validation by the platform. Dental Benefit Bid Requests 2008 will leverage data attributes from FIG. 1 pages: Health Purchaser Profile 1012, Health Purchaser Roster 1014, Health Purchaser Claims 1016, and will be properly aggregated with validation by the platform. Life Insurance Bid Requests 2010 will consider data attributes from FIG. 1 pages: Health Purchaser Profile 1012 and Health Purchaser Roster 1014, and will be properly aggregated with validation by the platform. Disability Insurance Bid Request 2012 will consider data attributes from FIG. 1 pages: Health Purchaser Profile 1012 and Health Purchaser Roster 1014, and will be properly aggregated with validation by the platform. Auto Bid Requests 2016 will consider data attributes from FIG. 1 pages: Health Purchaser Profile 1012 and Health Purchaser Roster 1014, and will be properly aggregated with validation by the platform. Pet Insurance Requests 2018 will be pulled employee-based forms only and will likely take place after enrollment meetings take place. Business Insurance Bid Requests 2020 leverage data attributes from FIG. 1 pages: Health Purchaser Profile 1012 and Health Purchaser Roster 1014, as well as one specific web form or table populated by the benefit administrator representing the employer. This information will be properly aggregated with validation by the platform. Based on flagged tokens captured within FIG. 1 page Health Purchaser Profile 1012 and the absence of Healthcare Claims data, prospect users can select to authorize Medical Information Bureau (MIB) and/or Parameds searches or engagement for Health Benefit Requests exclusively.

Turning to FIG. 3, illustrates a block diagram of an exemplary process 300 of health plan prospects exemplary that are “Direct Contract” to navigate on a platform in accordance with the disclosed principles. Thus, instead of being designated as a “Shopper”, the Inbound Prospect Data—Direct Contract 3000 includes prospects that identify themselves as interested parties in forming an Association Health Plan, HRA, or to migrate to self-insured. These prospects will also interact with the webpage via either form based manual keying of required and/or requested information, downloading form(s) independently and uploading completed forms via firewall protected interface. The ETL 3002 phase ensures web form mapping instructions and user error identification as the user interacts with the form(s). Users have the ability to download table formats and upload the completed forms. The ETL process will detect table identification and error handling instructions and propagate any errors back to the user. Staging and Data Categorization step 3004 takes place after each webpage form is completed and/or uploaded from the prospect. Based on responses from the prospect user, one to many of the following benefit requests will execute.

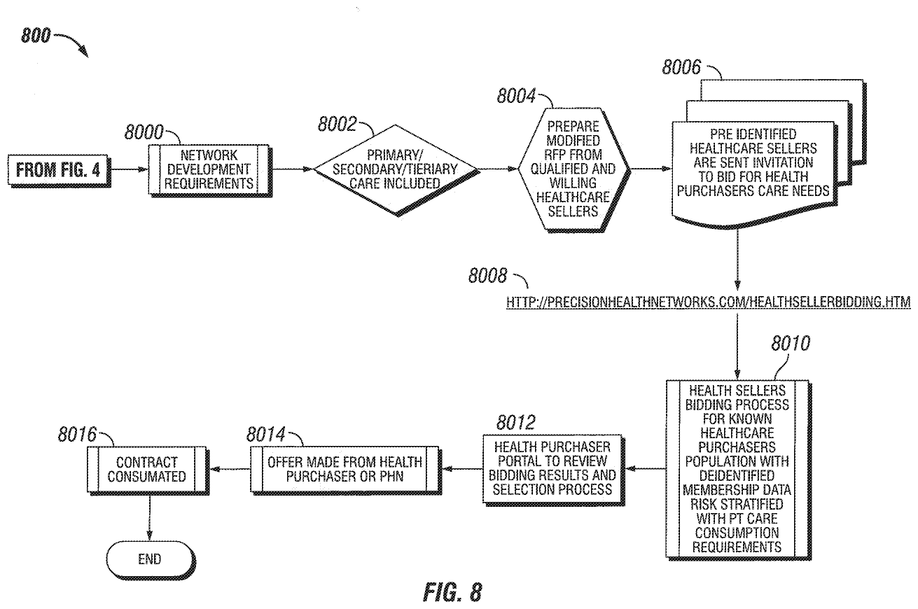

The United States Department of Labor has published changes to the definition of “Employer” under section 3 (5) of ERISA—Association Health Plan. This created clear requirements of minimum permissible standards that must be followed to qualify as Association Health Plan (AHP), including but not limited to: Commonality of Interest, Bona-fide governance, exist within the same ‘geographic region,’ etc. In FIG. 3, Marketing, Board Formation and Recruitment 3006 pertains to membership's willingness and availability to participate in the AHP. The AHP Network Development Process 3008 is illustrated in FIG. 8.

Based on the options selected by the healthcare service purchaser, health benefit bid requests 3010 will be activated to capture required information. Based on business rules, prospects must elect Healthcare Service Bid Requests or the platform will refuse to offer service. If for any reason a prospect elected NOT to complete step 1010 Health Purchaser Goals and/or 1012 Health Purchaser Profile, they will be referred back to these steps to proceed to any form of Health Benefit bids. Much of this information will be included within information illustrated in FIGS. 5, 6 and 7. Options for dental benefit bid requests 3012 are also provided, and information may be obtained from the information illustrated in FIGS. 5 and 6. Options for Life Insurance bid requests 3014 are also offered to the prospect, where all available types of life insurance will be available for selection. Options for Disability Insurance Bid Requests 3016 are also provided, where all available policies matching health purchaser profile 1012 (from FIG. 1) will be selected for bids. Options for Employee Retirement Bid requests 3018 are also provided, where all available policies matching health purchaser goals 1010 and health purchaser profile 1012 will be selected for bids. Options for Auto Bid requests 3020 will request specific information about vehicles, drivers, and coverages to be considered will be selected for bids. Options for Pet Insurance 3022 are based on number of employees. Options for Business Insurance Bid requests 3024 are based on specific questions or will be presented in-line for response from the prospect. At the successful completion of step 3024, the prospect will be provided a message about any missing required information (e.g. health claim history, authorization of MIB or missing company information) that a licensed agent will follow up with them with their bids as soon as the information becomes available.

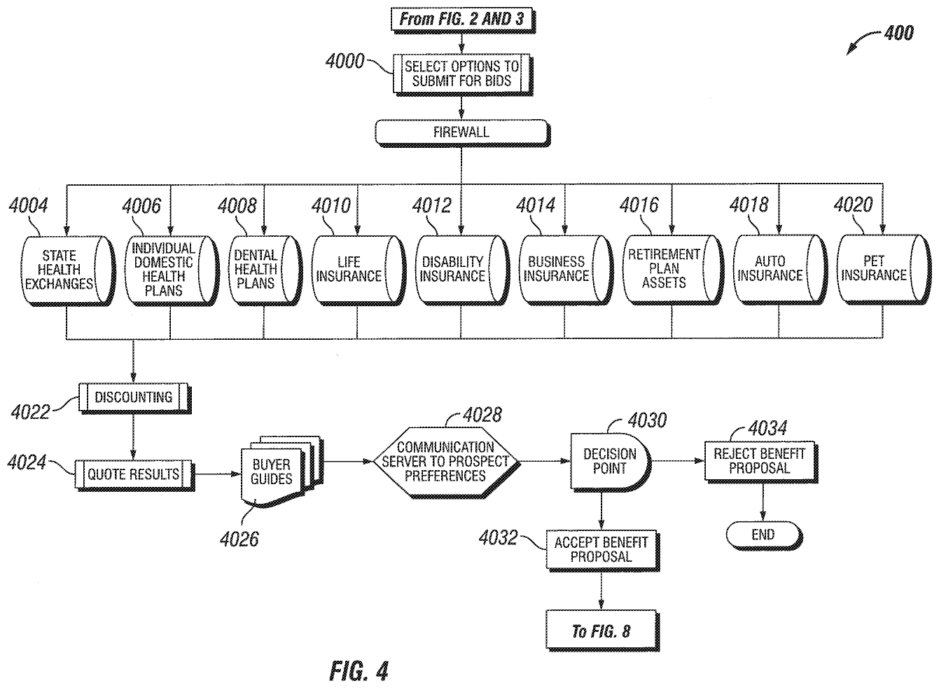

Behind the scenes, a decision tree 3026 of the platform will engage based on the health purchaser's election of Risk (e.g. Self-Insured) and internal platform rule sets. If either is true, the prospect purchaser elects self-insured option or the platform directs network development needs, the platform will begin either the claims assembly or MIB request to execute Risk Stratification 3028 and possible Parameds (underwriting) for Self-Insured health purchasers. Proprietary methods are used by the disclosed platform to combine Risk Stratification results with Patient/Member Consumption preferences 3030 and Health Risk Assessments made from the information provided in the pages illustrated in FIG. 1. Based on known comorbidities, treatment histories, gaps in care, utilization patterns and patient activation measures acknowledged from Risk Stratification software, associated medically necessary longitudinal care or treatment plans 3032 will be created for membership/patients. Preventative needs per U.S. Preventative Services Task Force (U.S. PSTF) will also be assembled and prioritized based on other known illness burdens reflected from risk stratification and utilization patterns. Primary Care Team Needs 3034 involves governance rules and preferred longitudinal care/treatment plans matched to membership/patients. Health Benefit Task level attribution to all members of the Interprofessional Primary Care Team actors by Actor role matched to state's-based scope of service. To accommodate Network Services or Provider network enrollment and bidding process this will introduce new tasks on FIG. 8. Step 3036 is an AND/OR logic depending on if the platform will be delegated network development responsibilities; if not—the process goes to FIG. 4, Step 4000. If so, then the platform will submit signed prospects Medical Information Bureau authorization 3038 if no claim histories are available. If the prospect elected Auto Insurance request—appropriate form documentation will be provided to the Department of Motor Vehicles 3040 for verification and onto licensed providers in state. Since the platform will not provide underwriting services, outsourcing to Parameds 3042 will provide underwriting services and forward on to platform-selected health plans licensed in state. When the platform is delegated responsibility for Primary Care network development, Wrapper Coverage 3044 (Basic Medical, Major Medical, Corridor Deductible, Dread Coverage, Short Term—Limited Duration Health Coverage, etc.) is engaged. When the platform is delegated and the prospect elects options of Reinsurance 3046 coverage, the platform will shop options and return bids to the prospect.

Moving on to FIG. 4 from either the process in FIG. 2 or FIG. 3, illustrated is an exemplary process 400 for a health plan purchaser to select coverage options for submission to healthcare sellers for bidding using a platform as disclosed herein. In particular, a platform licensed producer will review selected options for bid submittal 4000 and make any changes required. The platform licensed producer will validate a Primary Care Team Carve Out reconcile with options being submitted in the illustrated steps. Based on prospect preferences from FIG. 6 health insurance bid requests will be submitted to State Health Exchanges 4004 and/or Individual Domestic Health Plans 4006. Based on such prospect preferences, also submitted will be dental insurance bid requests 4008, life insurance bid requests 4010, disability insurance bid requests 4012, business insurance bid requests 4014, and retirement plan assets search bid requests 4016. A state licensed securities agent will fulfill all these requests and schedule follow up meeting with prospects to discuss options. Also based on prospects preferences, also submitted will be auto insurance search bid requests 4018 and pet insurance bid requests 4020. Platform policy bid compiler may also interrogate multi-policy discounting rules and submit bid requests at step 4022.

The platform will receive quote results at step 4024 from each submitted bid, and compile all for review of the licensed producers as appropriate. The platform will receive or follow-up with bidding health insurance companies to obtain Buyers Guides 4026 as required by state law for prospects. The platform will interrogate prospect preferences 4028 as well, and follow-up as appropriate. If the prospect elects electronic submission, email notification will be sent and followed up with phone call, for example, within 72 hours. If the prospect requested telephone conversation or on-site meeting with electronic delivery of quotes, those appointments will be set after the insurance quotes have been provided. A decision point 4030 will be in the prospect's hands after a licensed producer has reviewed all materials. The prospect may elect to Accept Benefit Proposal(s)—some or all—at step 4032, and thus the process will continue to FIG. 8. However, if the prospect elects' to Reject Benefit Proposal(s) at step 4034, the process ends and the prospect could be sent a Thank you letter/email.

Referring now to FIG. 8, illustrated is an exemplary process 800 for compiling and submitting health plan proposals for bidding by healthcare sellers, using a platform as disclosed herein. Network Development Requirements at step 8000 are compiled from platform direct server access and results are attributed, in step 8002, in Primary Care (Interprofessional Primary Care Team needs from FIG. 3—3034), Secondary Physician Specialist, other ambulatory and ancillary services, and Tertiary Care—acute, sub-acute and all other services based on risk stratification service needs, projected medical exacerbations, network preferences, projected cost, and member/patient care consumption preferences. The platform will format and prepare RFP's (Request for Proposals), at step 8004, for sending to qualified and willing healthcare service sellers-based inputs from Health Purchaser Goals (FIG. 5), Health Purchaser Profiles (FIG. 6), and Patient/Member Preferences (FIG. 7), as well as quality, outcome, cost and any other member/patient preference requirements that are contractually obligated. Pre-identified healthcare sellers are sent invitations to bid for Health Purchasers' care needs via the RFP requests. RFP requests may go out via U.S. Mail, other accelerated options, or encrypted email to healthcare service sellers network development resources. All interested healthcare service sellers will be provided secure access to the platform's website 8008 to review de-identified membership information, step 8010, included in the purchasers' request, healthcare purchaser profiles, goals, and requested services. Additionally, healthcare service sellers will have access to proposed contractual agreement to include fee schedules, other rates, bylaws, governance, quality and outcome expectations as well as incentive pool qualifications and budgeted pool distributions by contracted entity. These documents or records are all included in step 8010. Healthcare service sellers who are interested in bidding to cover prospects membership will submit positive responses to the platform via the website portal. The platform and the healthcare services purchaser can and will review interested qualified providers and select healthcare services sellers 8012 that best meet their membership needs at more affordable rates. Offers, at step 8014, or alternatively denial letters, from the platform and healthcare service purchasers with appropriate contractual documentation will be submitted via the portal to all bidders. The awarded healthcare service sellers will print and execute contractual agreements, scan and upload (or e-sign—based on state requirements and acceptance) via portal. Countersigned contracts will be published for all parties within the platform service in step 8016. The process may then end.

Turning now to FIG. 9, illustrated is a diagram having an exemplary process 900 for determining the primary care team and related provider costing for an exemplary purchaser's healthcare plan via a platform as disclosed herein. Primary Care Team assembly with proprietary task level activity-based costing 9000 leverages the Primary Care Team Needs from FIG. 3, step 3034, from healthcare service sellers/providers that have been contracted from FIG. 8, step 8016. Primary Care Team task level attribution 9002 is an underlying rule base for the incentive pool Health Barometer 9004 populated by Care Team Actor(s), which is based on task severity indicators. Interprofessional Primary Care Team appointment schedules will be populated with attributed task responsibilities and where possible updated to healthcare service sellers' EHR. Healthcare service purchasers will not be able to access membership's detailed care plan or treatment plans; however, transparency to task completion factors, non-compliance—provider or patient, over projected budgetary reasons, etc., will be available via the healthcare purchaser web site portal at step 9006.

If Proof of Insurability is required at step 9008, membership/patients/employees can access and complete. Healthcare service purchasers will interact with portal or licensed producer(s) to setup invoice processing/banking rules at step 9010. Health service purchasers can access all Policy Documents via the portal at step 9012. Healthcare service purchasers will sign or e-sign documents (based on state requirements and acceptance) via portal at step 9014. The platform will submit all executed agreements and required documents to health plan and/or other insurers at step 9016. All fully executed agreements and required documents will persist on portal, as shown at step 9018. The platform will announce Interprofessional Primary Care Team at step 9020, and schedule onsite visit(s) to meet membership/patients. Full details about 24/7/365 access to the team, telemedicine, and accountabilities will be discussed. The platform and the healthcare service purchaser will schedule human capital benefit promotions and employee engagement programs at step 9022, and afterwards the exemplary process is complete.

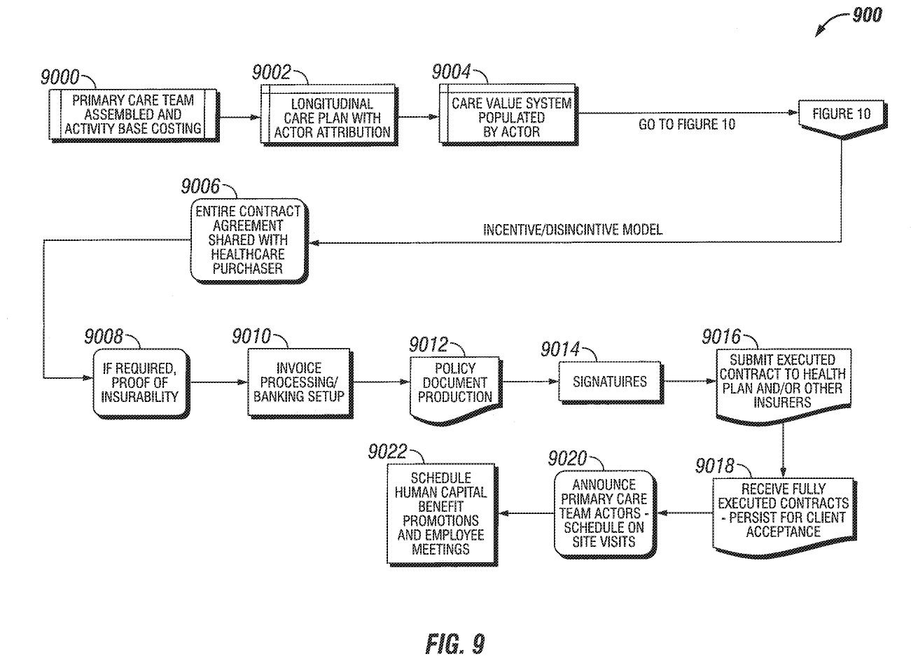

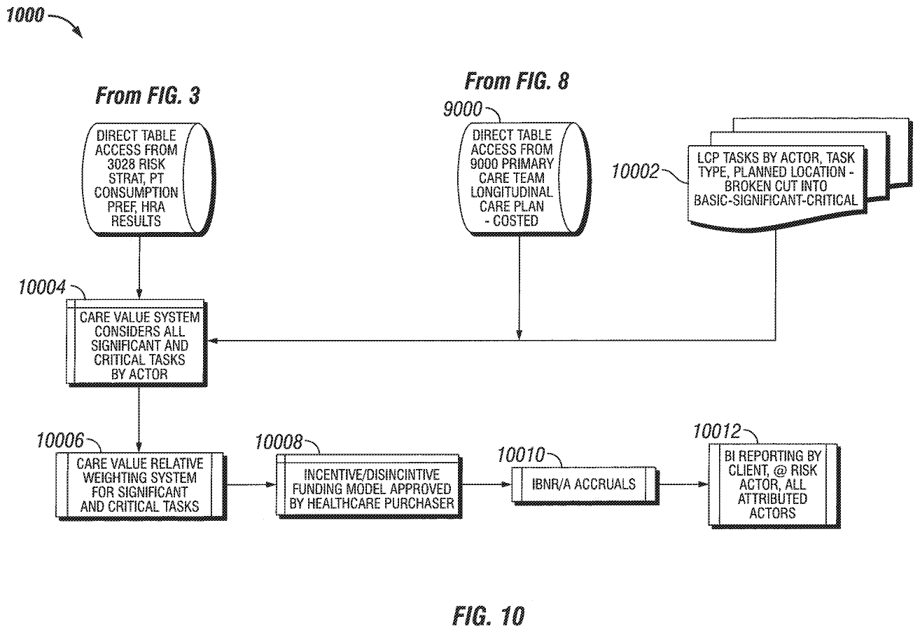

Inputs from both FIG. 3 and FIG. 9 are relevant in FIG. 10, which illustrates a diagram having an exemplary process 10000 for developing an exemplary incentive/disincentive model for using in health plan cost determining as performed on a platform implemented in accordance with the disclosed principles. From FIG. 3 is the direct table access from step 3028: Risk Stratification, Patient Care Consumption Preferences, as well as Health Risk Assessment results. This information is also joined with a direct table access from step 9000 of FIG. 9, where Interprofessional Primary Care Team LCP costing information becomes the baseline of known and projected cost to treat and manage known disease and illnesses. As such, these values are used to baseline for budgetary needs. LCP tasks by Actor, Task Type, planned location by Task severity at step 10002 is derived based on healthcare purchasers and patient preferences found in FIGS. 5, 6 and 7. Enterprise healthcare resource planning platform services assemble Care Team Actors based on qualifications meeting or exceeding healthcare services purchasers' standards (quality, outcome and cost), matched membership/patient preferences to ensure all planned services meet requirements as well as appropriate site for service.

At step 10004, Health Barometer consideration for Quality Outcome only factors “Significant” and “Critical” task types; however, Final Outcomes include all task types. The Health Barometer also includes a weighting at step 10006 for “Significant” and “Critical” tasks set in a governance process that is used within the incentive/disincentive pool calculations at step 10008. The benefit of activity-based costing full interprofessional primary care team tasks affords more accurate insurance Incurred but Not Recorded (known in industry as IBNR) and healthcare sellers' Net Revenue Accruals, as performed in step 10010. This process should also help healthcare service purchasers better plan for healthcare expenditures that are Planned Services. Incentive/Disincentive Pool funding during step 10008 is part of governance approvals between all parties including healthcare purchaser, healthcare seller, and the platform. Notable aforementioned task types and task weighting are considered first with step-down logic impacting if patient compliance is low and lastly financial performance of attributed tasks of care team. Step 10012 invokes longitudinal care plan tasks to each individual Care Team actor based on their role in the assigned Care Team alignment. This function is set to ensure adequate capacity exists for each Care Team actor to complete all assigned tasks.

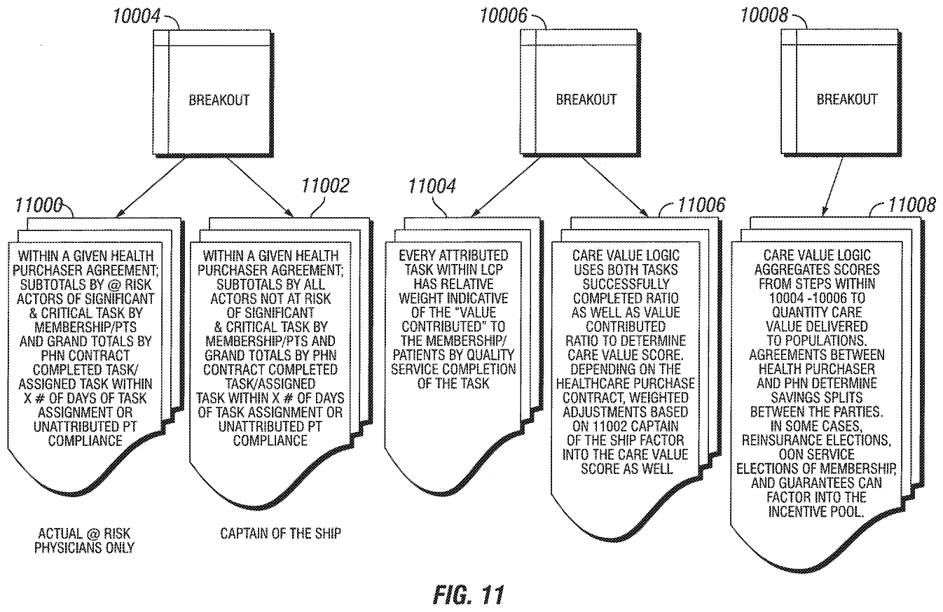

Turning now to FIG. 11, this figure provides more detail breakout from 3 steps of the process illustrated in FIG. 10. From FIG. 10, step 10004—detail and preferred incentive weights goes to at Risk Primary Care Physicians found in step 11000. These care team actors' “Significant” and “Critical” tasks attributed are aggregated in the denominator with consideration to “Grace Days” to complete as well as “Unattributed Patient Compliance”. The numerator is populated with all assigned and completed “Significant” and “Critical” tasks. Attributed Primary Care Physicians are also evaluated and rewarded based on “Captain of the Ship” provision providing preferred incentive, as found in step 11002. This measure considers all Interprofessional Care Team actors' “Significant” and “Critical” tasks at a Healthcare Servicer Purchaser level attributed and are aggregated in the denominator with consideration to “Grace Days” to complete as well as “Unattributed Patient Compliance”. The numerator is populated with all assigned and completed “Significant” and “Critical” tasks.

Further elaborated detail from FIG. 10, Step 10006 breakout can be found in step 11004 that defines within “Significant” and “Critical” tasks, relative weights that are indicative of how much healthcare value has been provided or derived, which contribute to the quality factor calculated at an Interprofessional Care Team actor level. Step 11006 also provides detail initially found in FIG. 10, step 10006. Care Value logic uses tasks completion ratio and value contributed ratio to determine a given care team actors' care value score. FIG. 10, step 10008, is further explained in 11008 as it defines that each step (based on care team actors' role) impact the overall Care Value logic, including at Risk physicians in step 11000, Captain of the Ship provision in step 11002 for all attributed Primary Care physicians, all member care team ‘Value Contributed’ calculations within step 11004, and Care Value completed task ratio and Care Value contributed ratio in step 11006 to provide final Care Value System scoring for all Care Team actors.

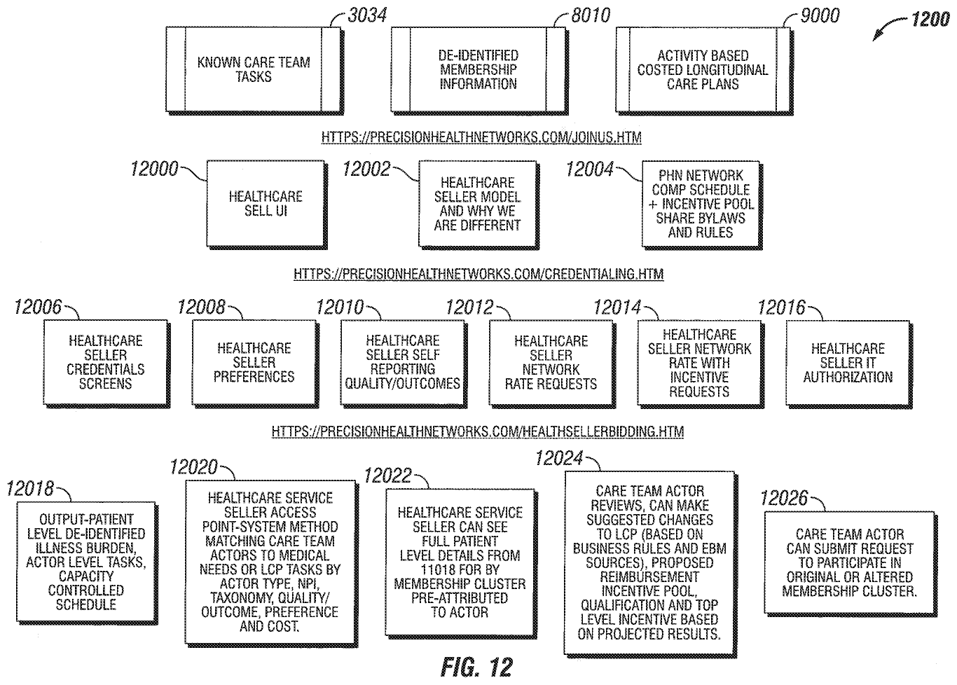

Turning now to FIG. 12, illustrated is a diagram 1200 showing various access portals available to healthcare sellers via a platform as disclosed herein. These portals include inputs from FIG. 3, step 3034 (Known Care Team Tasks, FIG. 8, step 8010 (De-identified membership), and FIG. 9, step 9000 (Activity based Costed Longitudinal Care Plans). Interprofessional Care Team Members have the ability to learn more about Precision Health Networks (or other appropriate entity implementing a platform in accordance with the disclosed principles), and the advantages of working with the platform and the healthcare service purchaser groups via exemplary url: https://precisionhealthnetworks.com/joinus.htm via a user interface at the launching point step 12000. Healthcare sellers can further understand and appreciate benefits of network inclusion in step 12002. Also, the Platform's Network, proposed compensation schedules, incentive pool design, benefit design, Interprofessional Care Team assembly, bylaws, governance and network rules in step 12004. If desired, Care Team applicants may elect to enter into marketplace bidding, however, they are required to first get credentialed at step 12006 via exemplary url: https://precisionhealthnetworks.com/credentialing.htm. Care Team applicants about their preferences of treatment and other Interprofessional Care Team applicants they would encourage to participate are found on step 12008. Healthcare seller's quality and outcomes information and planned results for bidding membership/patients are found on step 12010. Healthcare seller's BASE reimbursement or rate requests are entered in step 12012. Healthcare seller's BASE+INCENTIVE POOL reimbursement request in step 12014. Healthcare seller's IT rules, obligations, preferences and authorizations to bi-directional CCD/CCDA & ADT from their EHR and PAS in step 12016. Once all preceding steps are completed, healthcare service sellers can access membership information, actor assisted tasks, capacity-controlled schedule via step 12018. Further details on care team actor matching based on scope of service, actor type, NPI, Taxonomy, Quality/Outcome, preference and cost is available in step 12020. Full cluster attribution by Care Team actor is available in step 12022. Care team actors may review, edit attributes (based on governance and business rules from healthcare purchaser and the platform) for their personal elections within step 12024. Care team actors submit final request(s) to available alterable attributes and membership in step 12026.

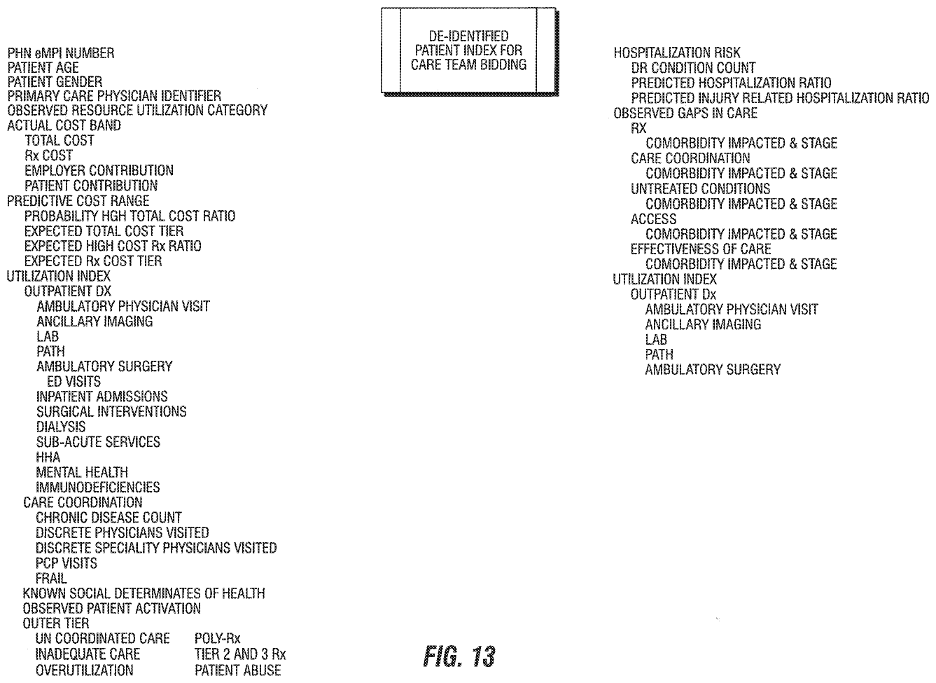

FIG. 13 illustrates exemplary lists of the de-identified membership/patient dataset available to Care Team applicants for Care Team applicants' bidding for health plans via a platform as disclosed herein. It should be noted that these exemplary lists are non-exhaustive, and thus no limitation to any particular information should be inferred.

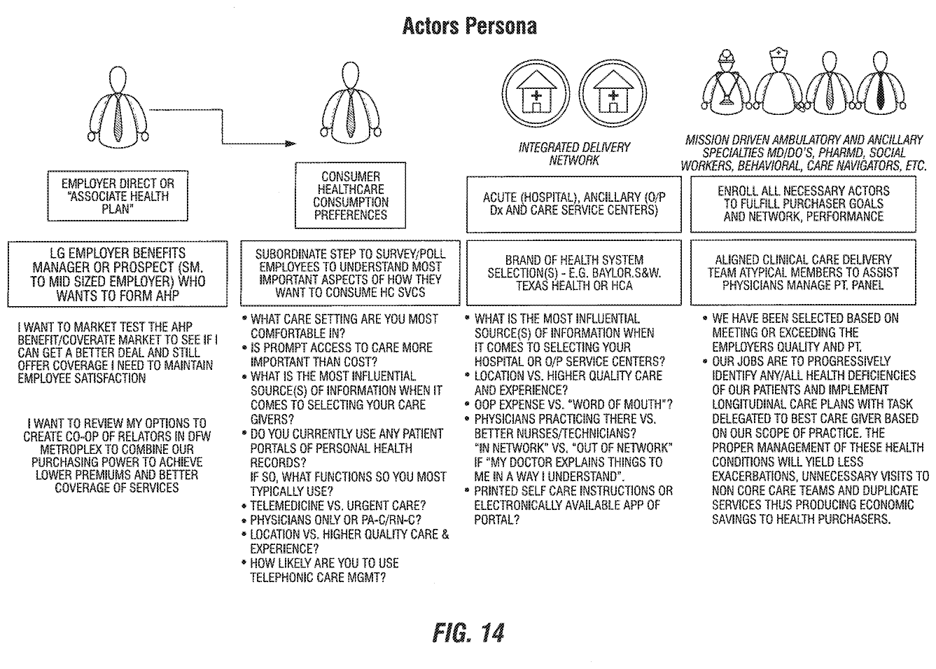

FIG. 14 sets forth the expectations and desires of key healthcare actors in a system or method implemented in accordance with the disclosed principles. The disclosed principles then provide for selection/acceptance by the health care purchaser via the platform of what becomes the Care Team that can provide at least the minimum coverage and quality of care desired by the purchaser's population. Thereafter, the selected Care Team is connected with the purchaser to provide both health care services and to develop long term health plans via the platform for each of the purchaser's population, as discussed herein.

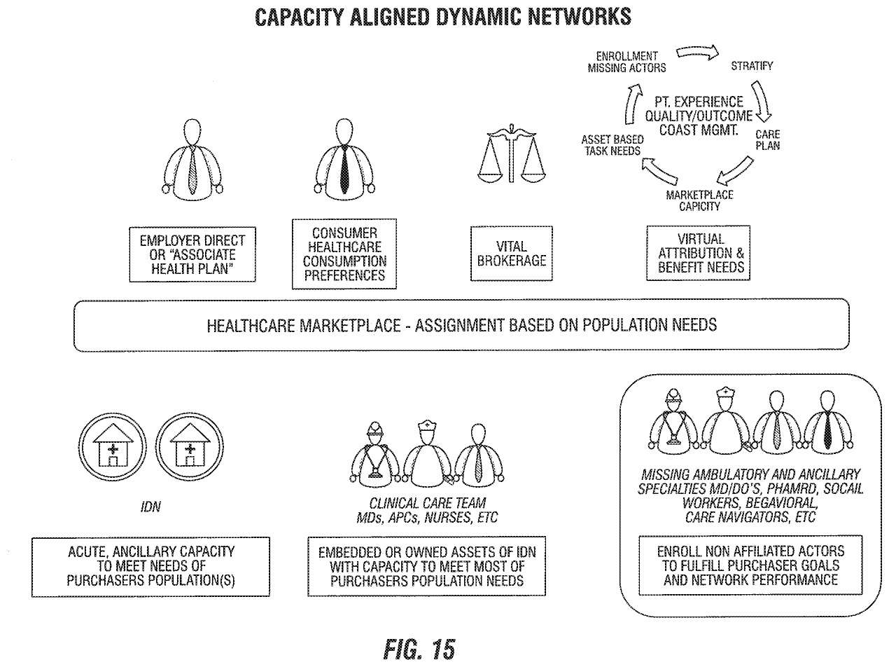

FIG. 15 illustrates the needs-based and capacity-based dynamic capabilities of a system or method (platform/network) implemented in accordance with the disclosed principles. The purchaser's population's needs (or alternatively an individual consumer/purchaser's needs) are used by the disclosed platform (i.e., providing a ‘healthcare marketplace’) to ensure the population's needs are met. As such, any bidding provider(s)/Care Team should have capacity internally, or have relationships with missing health providers, such as specialists, social workers, behavioral experts, etc. to fulfil the population's needs and goals. Alternatively, the disclosed platform can combine providers to create a Care Team that is capable of meeting all of the population's needs and goals, both immediate and for long term longitudinal care plans.

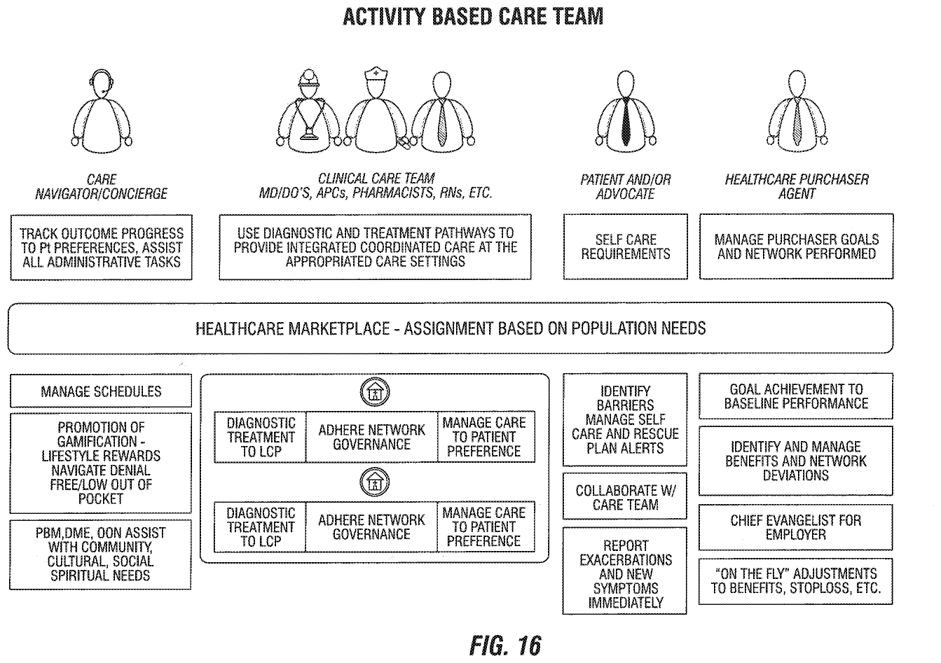

FIG. 16 illustrates exemplary actions of task level, activity-based care teams (actors) in a system or method implemented in accordance with the disclosed principles. Specifically, each actors or group of actors in the Care Team selected to fulfil a population's needs/goals continuously interacts with a platform as disclosed herein to provide a dynamic “healthcare marketplace.” The platform interacts with all of these Care Team actors regarding the needs and goals of each member of a population, and then interacts with each member individually to assist and ensure the member's immediate and ongoing healthcare needs are goals are being met. This may include, for example, the managing of the actual schedules of a member for appointments, healthcare goals, etc., but also to assist the member to find the specific providers capable of providing their needs and goals if one is not already part of the Care Team. This could include finding resources for the member's needs, assistance with in-network versus out-of-network providers, etc. The platform also helps identify for the member the current goal achievements, as well as barriers to their goals, such as missing appointments, failing to set an appointment with a provider for an identified healthcare need, and even reporting to current providers improvements or exacerbations in a member's condition or identification of possible new conditions based on current symptoms. Thus, the disclosed principles not only provide for a platform for determining a health plan purchaser's needs/goals and finding/matching providers with capacity, location, network-affiliation, costs, etc. that will meet those needs/goals, but also provides for the development of long term longitudinal health plans for the member, along with the continuous monitoring, maintenance, and when needed the modification of that plan for the member accomplished by dynamic, real-time interaction with both the member and the Care Team assigned to the member based on determined needs and goals.

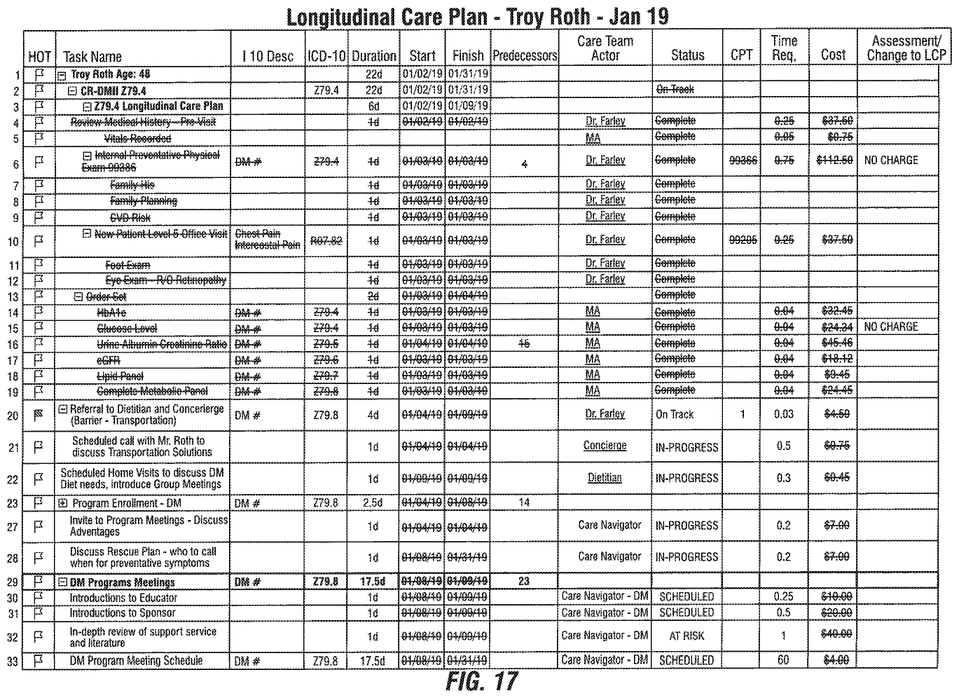

FIG. 17 illustrates an exemplary Longitudinal Care Plan (LCP) for a patient/member created by a system or method (platform) implemented in accordance with the disclosed principles. The illustrated portion of the Plan, which could be a spreadsheet accessible via the platform via a graphical user interface, sets forth exemplary healthcare-based appointments (recommended and/or actual) created for the patient, and includes insurance codes, dates, Care Team actors, status, time required, and costs for such healthcare appointments. As each appointment/step/need is achieved, the Plan spreadsheet may indicate completion, such as by the indicated strikethrough. Such notations may also be employed to illustrate expired/uncompleted tasks as well. The disclosed platform not only assists with the illustrated schedule, monitoring, billing, etc. of the member's needs, but also provides suggestions to address possible additional needs/goals determined based on information previously provided (e.g., results) to the platform. For example, if during a routine physical examination the results indicated the need for a dietary education or restrictions, the platform can then automatically create a referral to a dietician to address this determined need. Pre-determined “barriers” to achieving needs/goals can also be identified (such as the illustrated barrier of “transportation” to meet an appointment), as well as resources for overcoming such barriers. Collectively, the platform therefore facilitates determining healthcare needs and goals, identifying and connecting Care Team members to meet those needs/goals, determining the costs of treating the needs/goals, providing a schedule for meeting those needs/goals, monitoring the progress of achieving those needs/goals, determining whether the needs/goals are being met, identifying any additional or related needs/goals required to meet the original needs/goals, identifying barriers to achieving any of the needs/goals, and providing resources for overcoming those barriers. Of course, other information may also be provided.

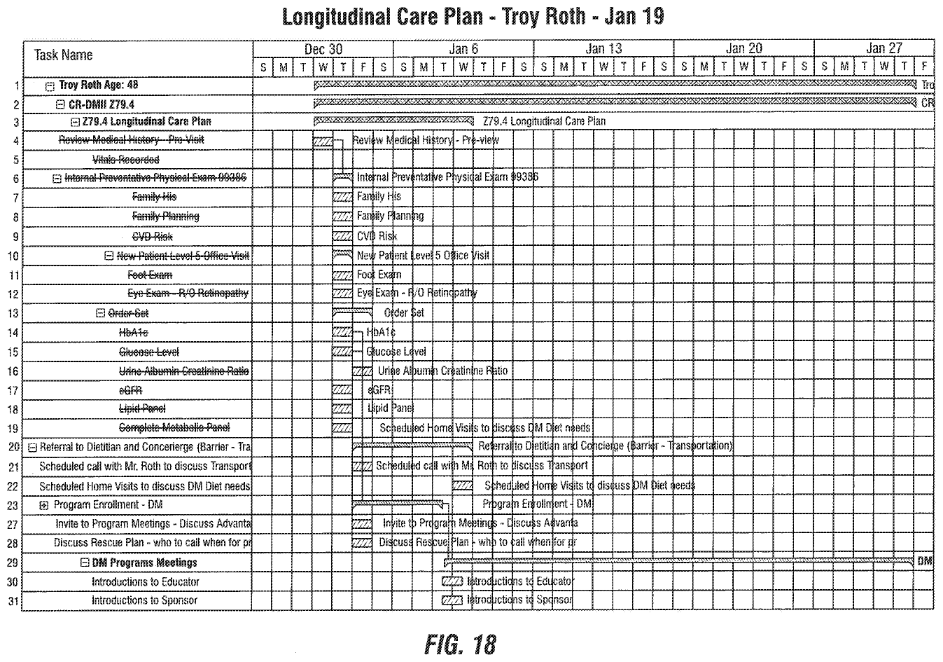

FIG. 18 illustrates additional time/date-based information for a portion of the exemplary Longitudinal Care Plan illustrated in FIG. 17 for a patient/member created by a system or method (network) implemented in accordance with the disclosed principles. This view of the Plan of the member helps provide a visual depiction of the time taken or needed for achieving the member's needs/goals. Of course, other information and detail may also be provided.

Improvements on Pre-Existing Inventions provided by the disclosed principles include but are not limited to:
Existing Insurance Brokerage capabilities including state-based Healthcare Marketplace.
Clinical Risk Stratification as a stand-alone solution.
Healthcare Provider Ratings based on informed consumer choice by Condition specific attributes.
Will include patient advocate or non-clinical caregiver experience and outcomes, including but not limited to: physical, social, emotional and Quality of Life indicators.
Specific attributes also include: shared decision making, service quality health plan benefits and coverages, Health Information Technology capabilities, access indicators and ancillary office staff helpfulness.
To the benefit of care team, Patient Activation Measures including patient compliance to follow treatment plan including barriers to fulfilling treatment plans, fill prescriptions and take medications as prescribed, make lifestyle changes, etc.
Accelerate migration to evidence-based medical treatments.
Built for purpose and preference networks enabling and warrantying healthcare services.

Accordingly, a key value provided by the disclosed principles is that the disclosed member-centric virtual brokerage helps create customizable value-based benefit and coverage plans for health purchasers who want to offer affordable health services primarily focused on delivering each employee and their families' health goals by aligning them with like-minded healthcare professionals and service providers that will reduce unnecessary spending and visits, while increasing employee satisfaction and retention. To meet this value, health purchasers contact the virtual broker (platform and related process) of the disclosed principles, and submit standard historic claim files and elect best means to collect patient preferences. The virtual broker will consume data to identify the populations' medical and preventative needs. A Longitudinal Care Plan and best service locations are created to set a baseline schedule and measurable care cost based on known requirements. The disclosed healthcare marketplace platform and related process will capacity-plan based on in network providers and service locations matched to purchasers' and patients' requirements, and if any tasks are unmet, the platform will solicit bids from other providers to match required services. Once the provider network is matched to membership—enrollment occurs and all care actors will receive their evidence-based care plan assignments.

### Exemplary Benefits of the Disclosed Principles

The disclosed principles provide for a platform and related process that provides a built-for-purpose healthcare benefit and coverage marketplace designed to ingress healthcare purchaser population(s), including past claim history, Medical Information Bureau or past medical information from other sources, such as Health Risk Assessments, including novel, newly introduced healthcare consumer preference questionnaires aimed to match how the consumer/purchaser wants to receive care. This information is used to attribute appropriate interprofessional primary and ancillary care teams designed to remove barriers to care delivery, affording those healthcare services purchasers to select from pre-population stratified benefit plans customized to meet memberships' disease burden, as well as their healthcare consumption preferences. Such benefit design yields expected outcome of more complete customized care delivery, as well as savings on healthcare expenditure to healthcare purchasers.

The disclosed principles also provide a purpose built, proactively assembled longitudinal care attribution model designed to specifically benefit healthcare purchasers who expect quality outcome-centric services that are matched to known disease burden and staging within their membership. Primary care team attribution is managed proactively via a capacity management system, thereby affording patients to preschedule all known needed services within the care plan upon enrollment to the healthcare purchaser's plan. Thus, the platform ensures not only that all of a patient's needs/goals will be met, but met within the shortest amount of time by the healthcare actors determined to be best suited to the patient, and at a competitive cost to the patient.

The disclosed principles also provide a platform to measure and calculate patient engagement and activation via Patient Health Engagement (PHE) Scale, Patient Activation Measure (PAM), and other proprietary artificial intelligence-capture bots learning capabilities to provide at least the automated benefits and features disclosed herein.

The disclosed principles also support proactive identification of care value risk, missing services, and provider attrition, leading to care gaps and members/patients with special needs that are not being met. A platform as disclosed herein identifies coverage gaps that are present to membership needs and allow riders or benefit redesign to meet all required care needs, such as short-term health plans that do not include ‘essential health benefits’. They also introduce flexibility on design for new alternative payment models (APM's) with reduced emphasis on administrative burdens with Fee-For-Service models.

The disclosed principles also provide for digital health applications (trusted biosensors, self-service devices, wearables and mobile applications) to promote engagement. In addition, the disclosed principles provide for simple, unburdened access and flow to all clinical settings, and convenience with balanced scorecard on value derived within selected benefit design. Moreover, the disclosed principles also provide for transparent transitions of care that enables all stakeholders' (patient and provider) ability to track progress in real time.

The disclosed principles also support the premise of treating patients at the most appropriate site-of-care (outcome/cost) via marketplace platform and proactive scheduler. The disclosed principles also provide for promotion of membership or patient educational knowledge about less than optimal care options selected. If health plan eligibility is submitted to a non-Tier 1 network provider and member/patient has other options, the platform will prospectively inquire with the member/patient about their choice and inform them about the potential higher patient portion.

Dynamic network performance attribution provided by a disclosed platform identifies care team and service center capacities and productivity via a patient/member-centric delivery model. The disclosed principles also provide for Care Team performance monitoring, and improves community health and wellness.

The disclosed principles also support health literacy/numeracy programs for health plan selection (how), Out-Of-Pocket projections based on disease stage and prior year costs, and consumption preference, chronic care, disease management and wellness programs for care team instructor led program administration.

The disclosed principles also afford ordering care team members and patients the ability to see patient and purchaser cost for all non-care plan-directed diagnostic tests, prescriptions, procedures within the disclosed dynamic network.

The disclosed principles also provide embedded machine learning to patient activation measures to best learn how to deliver care needs to each patient, and refine algorithms used to assess risk and attribute care teams. The disclosed principles also provide for creating and managing a clinically-supported machine learning solution to promote patient activation and active management. Measure will include minimum specification of: prescription adherence, self-care administration tasks completion based on agreed upon physician plan, lifestyle modification(s), and response to therapies. The disclosed principles also provide for the creation of first-to-market patient activation and enablement quality measures. The disclosed principles also provide for adaptability and function at work and home, while undertaking normal daily activities. A disclosed platform employs member demographics to utilization patterns—creating a “Shared Scoreboard” between patient and care team.

While various embodiments in accordance with the principles disclosed herein have been described above, it should be understood that they have been presented by way of example only, and not limitation. Thus, the breadth and scope of this disclosure should not be limited by any of the above-described exemplary embodiments, but should be defined only in accordance with any claims and their equivalents issuing from this disclosure. Furthermore, the above advantages and features are provided in described embodiments, but shall not limit the application of such issued claims to processes and structures accomplishing any or all of the above advantages.

Additionally, the section headings herein are provided for consistency with the suggestions under 37 C.F.R. 1.77 or otherwise to provide organizational cues. These headings shall not limit or characterize the invention(s) set out in any claims that may issue from this disclosure. Specifically, and by way of example, although the headings refer to a “Technical Field,” the claims should not be limited by the language chosen under this heading to describe the so-called field. Further, a description of a technology as background information is not to be construed as an admission that certain technology is prior art to any embodiment(s) in this disclosure. Neither is the “Summary” to be considered as a characterization of the embodiment(s) set forth in issued claims. Furthermore, any reference in this disclosure to “invention” in the singular should not be used to argue that there is only a single point of novelty in this disclosure. Multiple embodiments may be set forth according to the limitations of the multiple claims issuing from this disclosure, and such claims accordingly define the embodiment(s), and their equivalents, that are protected thereby. In all instances, the scope of such claims shall be considered on their own merits in light of this disclosure, but should not be constrained by the headings set forth herein.

Moreover, the Abstract is provided to comply with 37 C.F.R. § 1.72(b), requiring an abstract that will allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the claims reflect, inventive subject matter lies in less than all features of a single disclosed embodiment. Thus, the following claims are hereby incorporated into the Detailed Description, with each claim standing on its own as a separate embodiment.

What is claimed is:

1. A computer network-connected platform for developing and managing customizable healthcare plans for a population of healthcare purchasers, the platform comprising:
a purchaser interface for receiving, from a purchaser, purchaser information comprising purchaser goals for patient healthcare plans and purchaser profile, and patient information comprising patient(s) healthcare preferences, patient health profile(s), and patient(s) insurance claim history;
a processor, embodied in both hardware and software, configured to:
determine prospective care providers for a patient care team based on the received purchaser and patient information; and
compile requests for proposals for sending to the prospective care providers, the requests for proposals compiled based on the received purchaser and patient information;
a care provider interface for receiving, from prospective care providers, bids for providing healthcare benefits to the patient(s) based on the received purchaser and patient information;
wherein the processor is further configured to:
compile a list of potential care providers from the received bids from prospective care providers;
present the compiled list of potential care providers to the purchaser via the purchaser interface;
receive a selection of one or more of the potential care providers from the purchaser via the purchaser interface; and
create a provider care team for providing healthcare benefits to the patient(s) based on the received purchaser and patient information;
wherein the processor is further configured to:
create a long term care plan for each patient for meeting care goals to the patient based on patient healthcare needs, in accordance with the patient's patient preferences, through providers in the provider care team on an ongoing basis;
provide scheduling on behalf of the patient(s) for upcoming healthcare appointments with providers in the provider care team and related to meeting care goals;
determine recommended additional healthcare needs for the patient(s) based on current patient health information and care goals;
suggest healthcare providers and scheduling for said recommended healthcare needs to the patient(s); and
receive input from patient(s) via the purchaser interface for said scheduling.

2. A platform as recited in claim 1, wherein the processor is further configured to identify for the patient(s) achievements in care goals and barriers to achievement of care goals.

3. A platform as recited in claim 1, wherein the processor is further configured to provide reminders for upcoming appointments, notifications of missed appointments, failure to set an appointment for a care need, and notification for setting an appointment for a recommended care need.

4. A platform as recited in claim 1, wherein the processor is further configured to report to current providers of the care team improvements in patient condition, exacerbations in patient condition, and identification of possible new conditions based on current patient symptoms.

5. A platform as recited in claim 1, wherein the bids received from prospective care providers for providing healthcare benefits to the patient(s) comprise healthcare needs costs.

6. A platform as recited in claim 1, wherein the purchaser is the patient.

7. A platform as recited in claim 1, wherein the purchaser is a representative for a healthcare plan for a group of patients.

8. A platform as recited in claim 1, wherein the patient healthcare preferences are selected from the group consisting of:
if the patient has a primary care physician they trust;
if the patient would you like to stay with their primary care physician;
the patient's preferred healthcare setting;
what traits the patient wants most from their doctor;
how likely the patient is to use urgent care after office hours and weekends;
how likely the patient is to use telemedicine if it were easy and they saw their own doctor;
if the patient prefers brand name pharmaceuticals vs. generics;
what is the patient's normal tolerance for urgent appointment with their doctor;
what is the patient's tolerance for distance to their physician's office;
what is the patient's preferred departure point—home or workplace;
what is the patient's tolerance for distance to their pharmacy;
what is the patient's tolerance for distance to their hospital;
for medical expenses, how likely the patient is to be influenced by cost;
what a patient thinks of with regard to quality healthcare;
whether the patient is familiar with healthcare savings accounts;
whether the patient is familiar with flexible spending accounts;
whether the patient is familiar with healthcare reimbursement accounts;
whether the patient is familiar with medical savings accounts;
whether the patient would use other clinical experts if made available, such as: registered nurses for care navigators, pharmacists to help explain drug complications or symptoms, dieticians to help explain drug complications or symptoms, dieticians to help understand how diet influences health, disease specific health educators clinically trained to help understand lifestyle choices and how they impacts health or condition, wellness program leaders who can coach on lifestyle and cessation programs, behavioral clinicians who can help with personal and professional barriers, physician assistants and certified registered nurses in primary care setting,
whether the patient would use non-clinical experts if made available, such as: care concierges assisting with day-to-day administrative questions and issues, appointments and appointment changes, insurance questions such as requests for information, preapprovals, and denials, insurance benefits and coverage questions;
if the patient would you be willing to participate in programs such as: health risk assessments, biometric testing such as body mass index, blood pressure, cholesterol, glucose and/or fitness testing;
if the patient would be willing to enroll via a portal for appointments, alerts, and reminder messages; and
how likely the patient is to make lifestyle changes with regard to: eating, drinking, exercising, and nicotine use;

9. A platform as recited in claim 1, wherein the purchaser profile includes one or more of:
business zip code;
business classification (North American Industry Classification Scheme);
business' count of full-time employees;
count of proposed healthcare plan participants (including family);
prior 2 years' health insurance plans;
source of prior year health plan selection;
health benefits and/or disability options desired;
life insurance desired;
retirement options desired;
business insurance desired;
known hazards in the workplace;
adequate space on business property for employee benefit meeting;
private office on business property for clinical visits from care team; and
whether telemedicine options are acceptable;

10. A platform as recited in claim 1, wherein the purchaser goals for patient healthcare plans includes one or more of:
offer/obtain health insurance at reduced cost;
offer/obtain health insurance with less patient liability;
desire to form an association health plan for multiple member patients;
improved client services support for human resources and/or membership patients;
obtain more value per health insurance spend;
improved business intelligence or reporting;
desire to migrate from fully insured to self-insured;
if healthcare plan includes essential health benefits;
the inclusion of short term insurance options including pre-existing clauses;
if pharmacy benefit manager or other non-commercial options are acceptable;
individual or group spend expectations;
whether plans support lifestyle/wellness programs;
need for other coverages/services beside medical coverage, such as dental insurance, life insurance, disability insurance, business insurance, auto insurance, pet insurance, or retirement plans;
if narrow network coverage is acceptable;
recruit/retain skilled workforce for membership; and
reduce absenteeism in the workplace.

11. A platform as recited in claim 1, wherein the healthcare plan includes one or more of:
medical care benefits;
dental care benefits;
life insurance benefits;
disability insurance benefits;
retirement benefits;
pet insurance benefits; and
business insurance benefits.

12. A platform as recited in claim 1, wherein the processor is further configured to provide access of a patient's long term care plan to providers in the care team.

13. A method for developing and managing customizable healthcare plans for a population of healthcare purchasers, the method comprising:
receiving, from a purchaser via a purchaser interface connected to a computer network, purchaser information comprising purchaser goals for patient healthcare plans and purchaser profile, and patient information comprising patient(s) healthcare preferences, patient health profile(s), and patient(s) insurance claim history;
determining, with a processor embodied in both hardware and software, prospective care providers for a patient care team based on the received purchaser and patient information;
compiling, with the processor, requests for proposals for sending to the prospective care providers, the requests for proposals compiled based on the received purchaser and patient information;
receiving, from prospective care providers via a care provider interface connected to the computer network, bids for providing healthcare benefits to the patient(s) based on the received purchaser and patient information;
compiling, with the processor, a list of potential care providers from the received bids from prospective care providers;
presenting the compiled list of potential care providers to the purchaser via the purchaser interface;
receiving a selection of one or more of the potential care providers from the purchaser via the purchaser interface;
creating, with the processor, a provider care team for providing healthcare benefits to the patient(s) based on the received purchaser and patient information;
creating, with the processor, a long term care plan for each patient for meeting care goals to the patient based on patient healthcare needs, in accordance with the patient's patient preferences, through providers in the provider care team on an ongoing basis;
providing scheduling, with the processor, on behalf of the patient(s) for upcoming healthcare appointments with providers in the provider care team and related to meeting care goals;
determining, with the processor, recommended additional healthcare needs for the patient(s) based on current patient health information and care goals;
suggesting healthcare providers and scheduling for said recommended healthcare needs to the patient(s) with the processor; and
receiving input from patient(s) via the purchaser interface for said scheduling.

14. A method as recited in claim 13, further comprising identifying for the patient(s), using the processor, achievements in care goals and barriers to achievement of care goals.

15. A method as recited in claim 13, further comprising providing, using the processor, reminders for upcoming appointments, notifications of missed appointments, failure to set an appointment for a care need, and notification for setting an appointment for a recommended care need.

16. A method as recited in claim 13, further comprising reporting to current providers of the care team, using the processor, improvements in patient condition, exacerbations in patient condition, and identification of possible new conditions based on current patient symptoms.

17. A method as recited in claim 13, wherein the purchaser is the patient.

18. A method as recited in claim 13, wherein the purchaser is a representative for a healthcare plan for a group of patients.

19. A method as recited in claim 13, wherein the patient healthcare preferences are selected from the group consisting of:
if the patient has a primary care physician they trust;
if the patient would you like to stay with their primary care physician;
the patient's preferred healthcare setting;
what traits the patient wants most from their doctor;
how likely the patient is to use urgent care after office hours and weekends;
how likely the patient is to use telemedicine if it were easy and they saw their own doctor;
if the patient prefers brand name pharmaceuticals vs. generics;
what is the patient's normal tolerance for urgent appointment with their doctor;
what is the patient's tolerance for distance to their physician's office;
what is the patient's preferred departure point—home or workplace;
what is the patient's tolerance for distance to their pharmacy;
what is the patient's tolerance for distance to their hospital;
for medical expenses, how likely the patient is to be influenced by cost;
what a patient thinks of with regard to quality healthcare;
whether the patient is familiar with healthcare savings accounts;
whether the patient is familiar with flexible spending accounts;
whether the patient is familiar with healthcare reimbursement accounts;
whether the patient is familiar with medical savings accounts;
whether the patient would use other clinical experts if made available, such as: registered nurses for care navigators, pharmacists to help explain drug complications or symptoms, dieticians to help explain drug complications or symptoms, dieticians to help understand how diet influences health, disease specific health educators clinically trained to help understand lifestyle choices and how they impacts health or condition, wellness program leaders who can coach on lifestyle and cessation programs, behavioral clinicians who can help with personal and professional barriers, physician assistants and certified registered nurses in primary care setting,
whether the patient would use non-clinical experts if made available, such as: care concierges assisting with day-to-day administrative questions and issues, appointments and appointment changes, insurance questions such as requests for information, preapprovals, and denials, insurance benefits and coverage questions;
if the patient would you be willing to participate in programs such as: health risk assessments, biometric testing such as body mass index, blood pressure, cholesterol, glucose and/or fitness testing;
if the patient would be willing to enroll via a portal for appointments, alerts, and reminder messages; and
how likely the patient is to make lifestyle changes with regard to: eating, drinking, exercising, and nicotine use;

20. A method as recited in claim 13, wherein the purchaser goals for patient healthcare plans includes one or more of:
offer/obtain health insurance at reduced cost;
offer/obtain health insurance with less patient liability;
desire to form an association health plan for multiple member patients;
improved client services support for human resources and/or membership patients;
obtain more value per health insurance spend;
improved business intelligence or reporting;
desire to migrate from fully insured to self-insured;
if healthcare plan includes essential health benefits;
the inclusion of short term insurance options including pre-existing clauses;
if pharmacy benefit manager or other non-commercial options are acceptable;
individual or group spend expectations;
whether plans support lifestyle/wellness programs;
need for other coverages/services beside medical coverage, such as dental insurance, life insurance, disability insurance, business insurance, auto insurance, pet insurance, or retirement plans;
if narrow network coverage is acceptable;
recruit/retain skilled workforce for membership; and
reduce absenteeism in the workplace.
